(19) United States
(12) Patent Application Publication
(10) Pub. No.: US 2024/0375090 A1
(43) Pub. Date: Nov. 14, 2024

(54) CATALYST, PRODUCTION METHOD FOR CATALYST, AND PRODUCTION METHOD FOR alpha,beta-UNSATURATED ALDEHYDE AND/OR alpha,beta-UNSATURATED CARBOXYLIC ACID USING SAME

(71) Applicant: MITSUBISHI CHEMICAL CORPORATION, Tokyo (JP)

(72) Inventors: Kensuke NISHIKI, Tokyo (JP); Takuro WATANABE, Tokyo (JP)

(73) Assignee: MITSUBISHI CHEMICAL CORPORATION, Tokyo (JP)

(21) Appl. No.: 18/784,480

(22) Filed: Jul. 25, 2024

Related U.S. Application Data
(63) Continuation of application No. PCT/JP2023/001539, filed on Jan. 19, 2023.

(30) Foreign Application Priority Data
Jan. 27, 2022 (JP) 2022-011208

(51) Int. Cl. B01J 23/85; B01J 23/00; B01J 37/00; B01J 37/04; B01J 37/08; C07C 45/34; C07C 51/25

(52) U.S. Cl. CPC B01J 23/85; B01J 23/002; B01J 37/0045; B01J 37/04; B01J 37/08; C07C 45/34; C07C 51/25; B01J 2523/15; B01J 2523/53; B01J 2523/54; B01J 2523/68; B01J 2523/842; B01J 2523/845

(57) ABSTRACT

An object of the present invention is mainly to provide a catalyst with which an α,β-unsaturated aldehyde and/or an α,β-unsaturated carboxylic acid can be produced with a high selectivity. Provided is a catalyst used for producing, by an oxidation reaction of a hydrocarbon, a corresponding α,β-unsaturated aldehyde and/or α,β-unsaturated carboxylic acid, and the catalyst contains molybdenum, bismuth, and cobalt, and satisfies the following Formula (I-1): (x2−x1)/σ1≤1.5 (I-1) In Formula (I-1), x1, x2, and σ1 are values obtained by binarizing a reflected electron image of the catalyst, which is obtained using a scanning electron microscope (SEM) at an accelerating voltage of 15 kV, into black and white and subsequently performing an energy dispersive X-ray spectroscopy (EDS) analysis; x1 represents a bismuth concentration [% by mass] in black parts; x2 represents a bismuth concentration [% by mass] in white parts; and σ1 represents a standard deviation of the bismuth concentration in the black parts.

### CROSS-REFERENCE TO RELATED APPLICATION

This is a continuation of International Application PCT/JP2023/001539, filed on Jan. 19, 2023, and designated the U.S., and claims priority from Japanese Patent Application 2022-011208 which was filed on Jan. 27, 2022, the entire contents of which are incorporated herein by reference.

### TECHNICAL FIELD

The present invention mainly relates to a catalyst used for producing, by an oxidation reaction of a hydrocarbon, a corresponding α,β-unsaturated aldehyde and/or α,β-unsaturated carboxylic acid. More particularly, the present invention relates to a catalyst that is preferably used for producing, by an oxidation of propylene, isobutylene, t-butanol (hereinafter, also referred to as “TBA”), methyl t-butyl ether (hereinafter, also referred to as “MTBE”) or the like, a corresponding α, β-unsaturated aldehyde and/or α,β-unsaturated carboxylic acid.

### BACKGROUND ART

As a method of producing an α, β-unsaturated aldehyde and/or an α,β-unsaturated carboxylic acid, a method of performing an oxidation reaction of a hydrocarbon in the presence of a catalyst containing molybdenum and bismuth is known.

As the catalyst containing molybdenum and bismuth, for example, Patent Document 1 discloses a catalyst in which, when an SEM image of the catalyst, which is obtained using a scanning electron microscope (SEM) at an accelerating voltage of 15 kV, is binarized into black and white, the bismuth (Bi) concentration in white parts is higher than a value obtained by adding thereto 2.5 times the value of the standard deviation σ1 of the Bi concentration in black parts.

Further, Patent Document 2 discloses a catalyst which contains molybdenum, bismuth, and cobalt as indispensable components, and in which, in an X-ray diffraction pattern of the catalyst calcined at 550° C., a ratio (J) of a maximum peak intensity in a range of 2θ (X-ray diffraction angle)=28.4°±0.15° with respect to a maximum peak intensity in a range of 2θ=26.5°±0.3° is 36.0 to 57.5.

Moreover, Patent Document 3 discloses an oxide catalyst in which, in an X-ray diffraction pattern obtained using CuKα rays as an X-ray source, a ratio Ri=Pi/Ph of an intensity Pi of a β-Bi₂Mo₂O₉ diffraction peak (i) appearing at 2θ=27.76°±0.3° with respect to an intensity Ph of a CoMoO₄ diffraction peak (h) appearing at 2θ=26.5°±0.3° is 0.4≤Ri≤2.0.

### RELATED ART DOCUMENTS

### Patent Documents

[Patent Document 1] WO 2020/203606
[Patent Document 2] Japanese Unexamined Patent Application Publication No. 2020-151612
[Patent Document 3] Japanese Unexamined Patent Application Publication No. 2017-24009

### SUMMARY OF THE INVENTION

However, from the standpoint of the target product selectivity that is industrially required, even the catalysts disclosed in Patent Documents 1 to 3 do not necessarily have sufficient performance. Therefore, a further improvement in catalyst performance is demanded.

An object of the present invention is mainly to provide a catalyst with which an α,β-unsaturated aldehyde and/or an α,β-unsaturated carboxylic acid can be produced with a high selectivity. Another object of the present invention is to provide a method of producing an α,β-unsaturated aldehyde and/or an α,β-unsaturated carboxylic acid using the catalyst.

The present inventors intensively studied in view of the above-described problems, and consequently discovered that the problems can be solved by using a catalyst satisfying a specific bismuth concentration in a reflected electron image obtained by SEM, or a catalyst having a specific X-ray diffraction (XRD) pattern, thereby completing the present invention.

That is, the present invention encompasses the following.

[1] A catalyst used for producing, by an oxidation reaction of a hydrocarbon, a corresponding α,β-unsaturated aldehyde and/or α,β-unsaturated carboxylic acid,

wherein the catalyst contains molybdenum, bismuth, and cobalt, and satisfies the following Formula (I-1):

$$( x 2 - x 1 ) / σ 1 \leq 1.5 \qquad ( I - 1 )$$

(wherein, x1, x2, and σ1 are values obtained by binarizing a reflected electron image of the catalyst, which is obtained using a scanning electron microscope (SEM) at an accelerating voltage of 15 kV, into black and white and subsequently performing an energy dispersive X-ray spectroscopy (EDS) analysis; x1 represents a bismuth concentration [% by mass] in black parts; x2 represents a bismuth concentration [% by mass] in white parts; and σ1 represents a standard deviation of the bismuth concentration in the black parts).

[2] The catalyst according to [1], wherein the x1, x2, and σ1 satisfy the following Formula (I-2):

$$( x 2 - x 1 ) / σ 1 \leq 1 \qquad ( I - 2 )$$

[3] The catalyst according to [1] or [2], wherein the x1 and x2 satisfy the following Formula (I-3):

$$x 2 - x 1 \leq 3.5 \qquad ( I - 3 )$$

[4] The catalyst according to any one of [1] to [3], wherein the ad is 1.5 or more.

[5] The catalyst according to any one of [1] to [4], wherein, in an X-ray diffraction pattern, when the intensity of a maximum peak (peak A) in a range of 2θ (X-ray diffraction angle)=26.5°±0.3° is defined as IA and the intensity of a maximum peak (peak B) in a range of 2θ=28.0°±0.3° is defined as IB, a value of IB/IA is 0.08 or more.

[6] The catalyst according to [5], wherein the value of IB/IA is 0.08 to 0.5.

[7] A catalyst used for producing, by an oxidation reaction of a hydrocarbon, a corresponding α,β-unsaturated aldehyde and/or α,β-unsaturated carboxylic acid,
wherein
the catalyst contains molybdenum, bismuth, and cobalt, and
in an X-ray diffraction pattern, when the intensity of a maximum peak (peak A) in a range of 2θ (X-ray diffraction angle)=26.5°±0.3° is defined as IA and the intensity of a maximum peak (peak B) in a range of 2θ=28.0°±0.3° is defined as IB, a value of IB/IA is 0.08 to 0.5.

[8] The catalyst according to [6] or [7], wherein the value of IB/IA is 0.13 to 0.3.

[9] The catalyst according to any one of [1] to [8], wherein, in an X-ray diffraction pattern, when the intensity of a maximum peak (peak A) in a range of 2θ (X-ray diffraction angle)=26.5°±0.3° is defined as IA and the intensity of a maximum peak (peak C) in a range of 2θ (X-ray diffraction angle)=29.1°±0.3° is defined as IC, a value of IC/IA is 0.05 or more.

[10] The catalyst according to any one of [1] to [9], having a composition represented by the following Formula (II):

MoₐBi_{b}Fe_{c}Co_{d}XₑY_{f}Si_{g}Oₕ (II)
(wherein, Mo, Bi, Fe, Co, Si, and O represent molybdenum, bismuth, iron, cobalt, silicon, and oxygen, respectively; X represents at least one element selected from the group consisting of nickel, calcium, magnesium, niobium, tungsten, antimony, phosphorus, and titanium; Y represents at least one element selected from the group consisting of cesium, lithium, sodium, potassium, rubidium, and thallium; a, b, c, d, e, f, g, and h represent atomic ratios of the respective elements; a=12, b=0.01 to 3, c=0 to 5, d=1 to 12, e=0 to 8, f=0.001 to 2, and g=0 to 20; and h represents an atomic ratio of oxygen that is required for satisfying the valences of the respective elements).

[11] A catalyst production method, which is a method of producing the catalyst according to any one of [1] to [10], and includes the following steps (i) to (iii):
(i) the step of mixing a solution or slurry (liquid A) containing molybdenum and bismuth with a solution or slurry (liquid B) containing cobalt to prepare a slurry (liquid C);
(ii) the step of performing a dispersion treatment of the liquid C to prepare a slurry (liquid D); and
(iii) the step of drying the liquid D to obtain a dry product.

[12] The catalyst production method according to [11], wherein, in the step (ii), the liquid C is circulated for 5 minutes to 10 hours during the dispersion treatment of the liquid C.

[13] The catalyst production method according to [12], wherein, in the step (ii), the liquid C is circulated using a circulation pump.

[14] The catalyst production method according to any one of [11] to [13], wherein, in the step (ii), the liquid D is prepared by maintaining the liquid C at 60° C. or higher for at least 20 minutes after the dispersion treatment.

[15] A method of producing an α, β-unsaturated aldehyde and/or an α,β-unsaturated carboxylic acid, the method including the step of oxidizing a hydrocarbon in the presence of the catalyst according to any one of [1] to [10].

[16] A method of producing an α, β-unsaturated aldehyde and/or an α,β-unsaturated carboxylic acid, the method including the step of oxidizing a hydrocarbon in the presence of a catalyst produced by the method according to any one of [11] to [14].

[17] A method of producing an α, β-unsaturated carboxylic acid, the method including the step of oxidizing an α,β-unsaturated aldehyde produced by the method according to [15] or [16].

[18] A method of producing an α, β-unsaturated carboxylic acid ester, the method including the step of esterifying an α,β-unsaturated carboxylic acid produced by the method according to [17].

### Effects of the Invention

According to the present invention, a catalyst with which an α,β-unsaturated aldehyde and/or an α,β-unsaturated carboxylic acid can be produced with a high selectivity can be provided.

### BRIEF DESCRIPTION OF THE DRAWINGS

FIG. 1 is an X-ray diffraction pattern of the catalyst obtained in Example 1.

FIG. 2 is a reflected electron image of a catalyst surface, which was obtained by SEM in Example 1 (drawing substitute).

FIG. 3 is an image obtained by binarizing the reflected electron image of FIG. 2 into black and white (drawing substitute).

FIG. 4 is a reflected electron image of a catalyst surface, which was obtained by SEM in Example 2 (drawing substitute).

FIG. 5 is an image obtained by binarizing the reflected electron image of FIG. 4 into black and white (drawing substitute).

FIG. 6 is a reflected electron image of a catalyst surface, which was obtained by SEM in Comparative Example 1 (drawing substitute).

FIG. 7 is an image obtained by binarizing the reflected electron image of FIG. 6 into black and white (drawing substitute).

### MODE FOR CARRYING OUT THE INVENTION

Embodiments of the present invention will now be described; however, the present invention is not limited to the below-described embodiments.

In the present specification, those numerical ranges that are expressed with “to” each denote a range that includes the numerical values stated before and after “to” as the lower limit value and the upper limit value, respectively, and an expression “A to B” means a value that is A or more but B or less.

### [Catalyst]

The catalyst according to one embodiment of the present invention (hereinafter, also referred to as “the catalyst according to the first embodiment”) is a catalyst used for producing, by an oxidation reaction of a hydrocarbon, a corresponding (to the hydrocarbon) α,β-unsaturated aldehyde and/or α,β-unsaturated carboxylic acid, which catalyst contains molybdenum, bismuth, and cobalt, and satisfies the following Formula (I-1):

$$( x 2 - x 1 ) / σ1 \leq 1.5 \qquad ( I - 1 )$$

In Formula (I-1), x1, x2, and σ1 are values obtained by binarizing a reflected electron image of the catalyst, which is obtained using a scanning electron microscope (SEM) at an accelerating voltage of 15 kV, into black and white and subsequently performing an energy dispersive X-ray spectroscopy (EDS) analysis; x1 represents a bismuth concentration [% by mass] in black parts; x2 represents a bismuth concentration [% by mass] in white parts; and σ1 represents a standard deviation of the bismuth concentration in the black parts.

The catalyst according to another embodiment of the present invention (hereinafter, also referred to as “the catalyst according to the second embodiment”) is a catalyst used for producing, by an oxidation reaction of a hydrocarbon, a corresponding (to the hydrocarbon) α,β-unsaturated aldehyde and/or α,β-unsaturated carboxylic acid, which catalyst contains molybdenum, bismuth, and cobalt, and in which, in an X-ray diffraction pattern, when the intensity of a maximum peak (peak A) in a range of 2θ (X-ray diffraction angle)=26.5°±0.3° is defined as IA and the intensity of a maximum peak (peak B) in a range of 2θ=28.0°±0.3° is defined as IB, a value of IB/IA is 0.08 to 0.5.

By using either of the above-described catalysts, an α,β-unsaturated aldehyde and/or an α,β-unsaturated carboxylic acid can be produced with a high selectivity.

It is noted here that, such an “26.5°±0.3°” used herein means a range of “26.5°-0.3° to 26.5°+0.3°”.

In the present specification, the catalyst according to the first embodiment and the catalyst according to the second embodiment may be distinguished from each other to describe catalyst features; however, these features can be applied to both of the catalysts, including preferred conditions. For example, features of an X-ray diffraction pattern of the catalyst according to the second embodiment are applicable to the catalyst according to the first embodiment, and the features of the bismuth concentration of the catalyst according to the first embodiment are applicable to the catalyst according to the second embodiment. Further, where no special distinction is made between the catalyst according to the first embodiment and the catalyst according to the second embodiment, the term “catalyst” encompasses both of these catalysts.

Moreover, the expression “α, β-unsaturated aldehyde and/or α,β-unsaturated carboxylic acid” used herein can be paraphrased as “at least one selected from the group consisting of an α,β-unsaturated aldehyde and an α,β-unsaturated carboxylic acid”.

### <Bismuth Concentration in Reflected Electron Image>

The catalyst according to the first embodiment (in this section, also simply referred to as “catalyst”) satisfies the above-described Formula (I-1) when a reflected electron image of the catalyst, which is obtained using a scanning electron microscope (SEM) at an accelerating voltage of 15 kV, is binarized into black and white, and an energy dispersive X-ray spectroscopy (EDS) analysis is subsequently performed.

By using this catalyst, an α, β-unsaturated aldehyde and/or an α,β-unsaturated carboxylic acid can be produced with a higher selectivity. With regard to the reason for this, the present inventors presume as follows.

A reflected electron image reflects the atomic numbers of atoms existing on the surface of a sample and the irregular shape of the surface. In such a reflected electron image of the catalyst, those parts having a high concentration of high-atomic-number bismuth are emphasized in white due to the release of a large amount of reflected electrons from the sample. Therefore, when the reflected electron image of the catalyst is binarized into black and white, it can be said that white parts represent parts where bismuth is unevenly distributed, while black parts represent parts where the amount of bismuth is small. In an EDS analysis of the reflected electron image of the catalyst, satisfaction of the above-described Formula (I-1) indicates that the difference in bismuth concentration between the black parts and the white parts is small, i.e., bismuth is uniformly dispersed on the catalyst surface. It is believed that, when bismuth is not unevenly distributed and is uniformly dispersed on the catalyst surface in this manner, a selective oxidation reaction into an α,β-unsaturated aldehyde and/or an α,β-unsaturated carboxylic acid proceeds, and sequential reactions of these target products are inhibited, as a result of which the selectivity is further improved.

Further, in the EDS analysis of the reflected electron image of the catalyst, the above-described x1, x2, and σ1 preferably satisfy the following Formula (I-2):

$$( x 2 - x 1 ) / σ 1 \leq 1 \qquad ( I - 2 )$$

A lower limit of the value of (x2−x1)/σ1 is preferably more than 0, more preferably 0.05 or more, still more preferably 0.1 or more. An upper limit of the value of (x2−x1)/σ1 is preferably 0.9 or less, more preferably 0.8 or less, particularly preferably 0.7 or less.

Moreover, the above-described x1 and x2 preferably satisfy the following Formula (I-3):

$$x 2 - x 1 \leq 3.5 \qquad ( I - 3 )$$

In Formula (I-3), x1 and x2 each have the same meaning (are the same) as in Formula (I-1).

A lower limit of the value of x2−x1 is preferably more than 0, more preferably 0.2 or more, still more preferably 0.4 or more, particularly preferably 0.5 or more. An upper limit of the value of x2−x1 is preferably 3 or less, more preferably 2.5 or less, particularly preferably 2 or less, most preferably 1.5 or less.

A lower limit of the above-described σ1 is preferably 1.5 or more, more preferably 1.8 or more, still more preferably 2 or more. An upper limit of the σ1 is preferably 10 or less, more preferably 8 or less, still more preferably 6 or less.

As an SEM apparatus, for example, S-3400N manufactured by Hitachi, Ltd. can be used. As for the measurement conditions, a reflected electron image is obtained at an accelerating voltage of 15 kV. The current value is adjusted as appropriate such that an appropriate contrast is obtained within a range in which bright spots are not generated by electrification. As a measurement range, the magnification is set at a level where particles to be observed are included in a longitudinal visual field width. It is noted here that, as the particles to be observed, those particles having a size of at least 5 μm are selected.

The thus obtained reflected electron image can be binarized into black and white using, for example, Photos and Paint that are manufactured by Microsoft Corporation. Specifically, the reflected electron image saved in a bitmap format is opened in Photos manufactured by Microsoft Corporation, and an editing operation of adjusting the light to −80 is repeated three times. The resulting image is opened in Paint manufactured by Microsoft Corporation and saved as a monochromatic image, whereby the image can be binarized into black and white. In this process, the contrast and the brightness may be adjusted so as to emphasize light and shade.

The image thus binarized into black and white is analyzed by energy dispersive X-ray spectroscopy (EDS) to measure the bismuth concentration. As for the EDS measurement conditions, the accelerating voltage is set at 15 kV, and the current value is set as high as possible within a range that does not cause damage to a sample. Elements to be measured are those elements (including bismuth) that have a higher atomic number than oxygen and a greater number of moles than bismuth in the catalyst. The analysis time per measurement spot is set such that the count number of all measured elements is 5,000 or more and the count number of bismuth is 10,000 or more. In the image binarized into black and white, 10 spots of white parts are selected and point-analyzed, and x2 is calculated from an average value of the 10 spots. Further, with regard to black parts, an arbitrary spot is selected in the vicinity of the respective white parts measured just before, and 10 of these spots are point-analyzed in the same manner, after which x1 and σ1 are calculated from an average value and a standard deviation of the 10 spots.

Examples of a method for obtaining a catalyst that satisfies the above-described Formulae (I-1) to (I-3) include a method of producing a catalyst by a method including the below-described steps (i) to (iii).

### <X-Ray Diffraction Pattern of Catalyst>

In an X-ray diffraction (XRD) pattern of the catalyst according to the second embodiment (in this section, also simply referred to as “catalyst”), the value of IB/IA is 0.08 to 0.5. Further, when the catalyst satisfies the above-described Formula (I-1), in the X-ray diffraction pattern of the catalyst, a lower limit of IB/IA is preferably 0.08 or more, and an upper limit of IB/IA is preferably 0.5 or less.

The lower limit of IB/IA is more preferably 0.1 or more, still more preferably 0.11 or more, particularly preferably 0.12 or more, most preferably 0.13 or more. The upper limit of IB/IA is more preferably 0.4 or less, still more preferably 0.3 or less.

When the catalyst satisfies the above-described range of IB/IA, an α, β-unsaturated aldehyde and/or α, β-unsaturated carboxylic acid can be produced with a high selectivity. With regard to the reason for this, the present inventors presume as follows.

In the X-ray diffraction pattern, the peak A is a diffraction peak attributed to the (002) plane of a cobalt-containing crystalline phase, and the peak B is a diffraction peak attributed to the (032) plane of a bismuth-containing crystalline phase. The bismuth-containing crystalline phase is believed to function as an active site in an oxidation reaction of a hydrocarbon. It is believed that, therefore, when the value of IB/IA, which is an intensity ratio of the peak A and the peak B, is 0.08 or more, i.e., when the bismuth-containing crystalline phase is contained in a large amount, a selective oxidation reaction into an α,β-unsaturated aldehyde and/or an α,β-unsaturated carboxylic acid proceeds, as a result of which the selectivity of the α,β-unsaturated aldehyde and/or the α,β-unsaturated carboxylic acid is improved. Further, when the value of IB/IA is 0.5 or less, the bismuth-containing crystalline phase is not formed excessively, and exists at an appropriate proportion. It is believed that, therefore, sequential oxidation of the resulting α,β-unsaturated carboxylic acid is inhibited, so that a reduction in the selectivity of the α,β-unsaturated aldehyde and/or the α,β-unsaturated carboxylic acid is inhibited.

Examples of a method for obtaining a catalyst satisfying the above-described IB/IA include a method of producing a catalyst by a method including the below-described steps (i) to (iii).

Moreover, when the intensity of a maximum peak (peak C) in a range of 2θ (X-ray diffraction angle)=29.1°±0.3° is defined as IC, the value of IC/IA is 0.05 or more. A lower limit of IC/IA is more preferably 0.07 or more, still more preferably 0.09 or more, particularly preferably 0.1 or more. An upper limit of IC/IA is not particularly limited; however, it is preferably 0.3 or less, more preferably 0.25 or less.

The peak C is a diffraction peak attributed to the (−104) plane of the bismuth-containing crystalline phase. It is believed that, therefore, similarly to the above-described case of IB/IA, when the value of IC/IA, which is an intensity ratio of the peak A and the peak C, is 0.05 or more, i.e., when the bismuth-containing crystalline phase is contained in a large amount, a selective oxidation reaction into an α,β-unsaturated aldehyde and/or an α,β-unsaturated carboxylic acid proceeds, as a result of which the selectivity of the α,β-unsaturated aldehyde and/or the α,β-unsaturated carboxylic acid is improved.

For the measurement of X-ray diffraction pattern, for example, an X-ray diffractometer X'Pert Pro MRD manufactured by Malvern Panalytical Ltd. can be used. The measurement is performed using CuKα rays (A=0.154 nm) at an output of 45 kV and 40 mA in a measurement range of 5° to 60°. The thus obtained X-ray diffraction pattern is corrected using a straight line connecting 2θ=20° and 2θ=50.2° as a baseline to determine the intensity of each peak.

### <Composition of Catalyst>

As described above, the catalyst according to the present embodiment contains molybdenum, bismuth, and cobalt. From the standpoint of improving the target product selectivity, when the number of molybdenum atoms is 12, a ratio of the number of bismuth atoms is preferably 0.01 to 3. A lower limit of the ratio of the number of bismuth atoms is more preferably 0.03 or higher, still more preferably 0.05 or higher. An upper limit thereof is more preferably 2 or lower, particularly preferably 1 or lower. From the same standpoint, when the number of molybdenum atoms is 12, a ratio of the number of cobalt atoms is preferably 1 to 12. A lower limit of the ratio of the number of cobalt atoms is more preferably 2 or higher, still more preferably 3 or higher. An upper limit thereof is more preferably 11 or lower, more preferably 10 or lower.

The catalyst according to the present embodiment may also contain, as other element, for example, iron, silicon, oxygen, nickel, calcium, magnesium, niobium, tungsten, antimony, phosphorus, titanium, cesium, lithium, sodium, potassium, rubidium, or thallium. From the standpoint of improving the target product selectivity, the catalyst preferably contains iron, and preferably contains at least one element selected from the group consisting of cesium, lithium, sodium, potassium, rubidium, and thallium.

Further, the catalyst according to the present embodiment may have a carrier for supporting the above-described elements. Examples of the carrier include, but not particularly limited to, silica, alumina, silica-alumina, magnesia, titania, and silicon carbide. Thereamong, silica is preferred for the inhibition of a reaction of the carrier itself. In the present specification, when a carrier is used in a catalyst, the carrier is also deemed as a component of the catalyst.

From the standpoint of improving the target product selectivity, the catalyst according to the present embodiment particularly preferably has a composition represented by the following Formula (II). The catalyst may also contain a small amount of an element that is not included in the following Formula (II).

MoₐBi_{b}Fe_{c}Co_{d}XₑY_{f}Si_{g}Oₕ (II)

In Formula (II), Mo, Bi, Fe, Co, Si, and O represent molybdenum, bismuth, iron, cobalt, silicon, and oxygen, respectively; X represents at least one element selected from the group consisting of nickel, zinc, chromium, lead, manganese, calcium, magnesium, niobium, silver, barium, tin, tantalum, tungsten, antimony, phosphorus, boron, sulfur, selenium, tellurium, cerium, and titanium; Y represents at least one element selected from the group consisting of cesium, lithium, sodium, potassium, rubidium, and thallium; a, b, c, d, e, f, g, and h represent atomic ratios of the respective elements; a=12, b=0.01 to 3, c=0 to 5, d=1 to 12, e=0 to 8, f=0.001 to 2, and g=0 to 20; and h represents an atomic ratio of oxygen that is required for satisfying the valences of the respective elements.

In the above-described Formula (II), from the standpoint of improving the target product selectivity, it is preferred that b, c, d, e, f, and g satisfy the following conditions.

A lower limit of b is preferably 0.01 or more, more preferably 0.03 or more, still more preferably 0.05 or more. An upper limit of b is preferably 3 or less, more preferably 2 or less, particularly preferably 1 or less.

A lower limit of c is preferably 0.01 or more, more preferably 0.1 or more, still more preferably 0.5 or more. An upper limit of c is preferably 4.5 or less, more preferably 4 or less, still more preferably 3.5 or less.

A lower limit of d is preferably 0.01 or more, more preferably 0.1 or more, still more preferably 1 or more, particularly preferably 3 or more. An upper limit of d is preferably 10 or less, more preferably 9 or less.

A lower limit of e is preferably 0.1 or more, more preferably 0.2 or more, still more preferably 0.5 or more. An upper limit of e is preferably 6 or less, more preferably 4 or less.

A lower limit of f is preferably 0.05 or more, more preferably 0.1 or more, still more preferably 0.2 or more. An upper limit of f is preferably 1.8 or less, more preferably 1.6 or less, still more preferably 1.4 or less.

An upper limit of g is preferably 15 or less, more preferably 10 or less.

In the present specification, the atomic ratio of each element is defined as a value determined by analyzing the components of the catalyst dissolved in hydrochloric acid by ICP emission spectrometry.

### [Catalyst Production Method]

The catalyst according to the above-described embodiment can be produced in accordance with any known catalyst production method as long as the conditions of the catalyst according to the first embodiment or the conditions of the catalyst according to the second embodiment are satisfied. However, the catalyst is preferably produced by the catalyst production method according to another embodiment of the present invention, which includes the following steps (i) to (iii):
(i) the step of mixing a solution or slurry (liquid A) containing molybdenum and bismuth with a solution or slurry (liquid B) containing cobalt to prepare a slurry (liquid C);
(ii) the step of performing a dispersion treatment of the slurry (liquid C) to prepare a slurry (liquid D); and
(iii) the step of drying the liquid D to obtain a dry product.

The catalyst production method according to the present invention may further include the below-described calcination step and molding step.

These steps will now each be described in detail.

<Step (i)>

In the step (i), a slurry (liquid C) is prepared by mixing a solution or slurry (liquid A) containing molybdenum and bismuth with a solution or slurry (liquid B) containing cobalt.

The liquid A is prepared by mixing raw material compounds of molybdenum and bismuth with a solvent. It is preferred that raw material compounds of the elements X and Y in the above-described Formula (II) be further mixed. The amount of each raw material compound to be used may be adjusted as appropriate such that a desired catalyst composition is obtained. Further, the amount of the solvent to be used is not particularly limited; however, it is preferably 70 to 400 parts by mass with respect to a total of 100 parts by mass of the raw material compounds.

The liquid B is prepared by mixing a raw material compound of cobalt with a solvent. It is preferred that a raw material compound of iron be further mixed. The amount of each raw material compound to be used may be adjusted as appropriate such that a desired catalyst composition is obtained. Further, the amount of the solvent to be used is not particularly limited; however, it is preferably 30 to 230 parts by mass with respect to a total of 100 parts by mass of the raw material compounds.

The raw material compounds are each not particularly limited and, for example, oxides, chlorides, hydroxides, sulfates, nitrates, carbonates, ammonium salts, or acetates of the respective elements, or mixtures thereof may be used. Examples of a molybdenum raw material include ammonium paramolybdate, molybdenum trioxide, and molybdenum chloride, among which ammonium paramolybdate is preferred. Examples of a bismuth raw material include bismuth nitrate, bismuth oxide, and bismuth subcarbonate, among which bismuth oxide is preferred. Examples of a cobalt raw material include cobalt nitrate, cobalt hydroxide, cobalt oxide, and cobalt chloride, among which cobalt nitrate is preferred. These raw material compounds may be used singly, or in combination of two or more kinds thereof.

The solvent preferably contains water and, more preferably, not less than 50% by mass of the whole solvent is water. The solvent may also contain an organic solvent, such as alcohol or acetone.

By mixing the liquid A and the liquid B, a slurry (liquid C) can be prepared.

<Step (ii)>

In the step (ii), a slurry (liquid D) is prepared by a dispersion treatment of the slurry (liquid C) obtained in the above-described step (i). By performing the dispersion treatment of the liquid C and thereby uniformly dispersing solid components in the liquid C, bismuth is uniformly dispersed on the surface of the resulting catalyst, so that a catalyst satisfying the above-described Formula (I-1) can be obtained in a favorable manner. In addition, the formation of a bismuth-containing crystalline phase is facilitated, so that a catalyst having a IB/IA value of 0.08 or more in its X-ray diffraction pattern can be obtained in a favorable manner.

As a method of performing the dispersion treatment of the liquid C, for example, a method of treating the liquid C in a container using a high-pressure-type, ultrasonic-type, or stirring-type homogenizer may be employed, and it is preferred to use a stirring-type homogenizer.

In the dispersion treatment of the liquid C, it is preferred to circulate the liquid C for 5 minutes to 10 hours. By this, bismuth contained in the liquid C is dispersed more uniformly. It is noted here that the term “circulate” used herein refers to an operation of discharging the liquid C to the outside of the container and then bringing the liquid C back into the container. A lower limit of the duration of the dispersion treatment is preferably 10 minutes or longer, more preferably 30 minutes or longer, still more preferably 60 minutes or longer. An upper limit of the duration is preferably 9 hours or shorter, more preferably 8 hours or shorter, still more preferably 7 hours or shorter. Further, the dispersion treatment is preferably performed with the temperature of the liquid being maintained at 20 to 90° C. From the standpoint of efficiently and uniformly dispersing bismuth, it is preferred to use a circulation pump for circulating the liquid C. As the circulation pump, for example, a centrifugal pump, a mixed-flow pump, an axial-flow pump, or a volumetric pump can be used.

After the dispersion treatment of the liquid C, the resulting slurry is preferably retained at 60° C. or higher for at least 20 minutes to prepare a liquid D. By this, the formation of a bismuth-containing crystalline phase is further facilitated. A lower limit of the retention temperature is preferably 70° C. or higher, more preferably 80° C. or higher. An upper limit of the retention temperature is not higher than the boiling point of the solvent, and it is preferably 150° C. or lower, more preferably 130° C. or lower. A lower limit of the retention time is preferably 30 minutes or longer, more preferably 40 minutes or longer. An upper limit of the retention time is preferably 4 hours or shorter, more preferably 2 hours or shorter. Further, during the retention of the slurry, the slurry is preferably stirred using a rotary blade stirrer or a magnetic stirrer.

<Step (iii)>

In the step (iii), a dry product is obtained by drying the liquid D obtained in the above-described step (ii). A method of drying the liquid D is not particularly limited, and examples thereof include a drying method using a box dryer, a drying method using a spray dryer, a drying method using a slurry dryer, a drying method using a drum dryer, and a method of pulverizing an aggregated solid obtained by evaporating the liquid D to dryness. Drying conditions are not particularly limited and, for example, when a box dryer is used, the drying is preferably performed at a temperature of 30 to 150° C. When a spray dryer is used, its inlet temperature and outlet temperature are preferably set at 100 to 500° C. and 100 to 300° C., respectively.

The dry product obtained in the step (iii) exhibits a catalyst performance and can thus be used as a catalyst; however, the dry product is preferably further subjected to the below-described calcination and/or molding since the performance as a catalyst is thereby improved. In the present embodiment, a catalyst obtained after the calcination and a catalyst obtained after the molding are collectively referred to as “catalyst”.

### <Calcination Step>

The dry product obtained in the above-described step (iii) may, in some cases, contain salts of nitric acid and the like that originate from the raw material compounds and the like. Therefore, in order to eliminate such salts, it is preferred to calcinate the dry product and obtain a calcination product. This calcination can be performed after obtaining a molded product by the below-described molding step; however, from the standpoint of the catalyst strength, the calcination is preferably performed prior to the molding step. Further, the calcination may be performed only once, or may be performed in plural separate occasions in combination with the below-described molding step. It is preferred to first perform primary calcination of the dry product for the purpose of eliminating the salts, subsequently perform the below-described molding step, and then perform secondary calcination for the formation of a final catalyst active site structure. Alternatively, the primary calcination and the secondary calcination may be performed before performing the molding step.

The calcination is preferably performed in a stream of an oxygen-containing gas such as air, or in a stream of an inert gas such as nitrogen, carbon dioxide, helium, or argon. From the standpoint of the selectivity of the resulting catalyst, the temperature of each calcination process is preferably 200 to 700° C., and it is more preferred that a lower limit thereof be 250° C. or higher and an upper limit thereof be 600° C. or lower. The duration of each calcination process is selected as appropriate in accordance with the target catalyst; however, from the standpoint of the selectivity of the resulting catalyst, it is preferably 10 minutes to 10 hours, and it is more preferred that a lower limit thereof be 1 hour or longer and an upper limit thereof be 10 hours or shorter. It is noted here that the “duration of calcination” refers to a period of continuing the calcination after a prescribed calcination temperature is reached.

In a case where the dry product is subjected to the primary calcination, the below-described molding step, and then the secondary calcination, the temperature of the primary calcination is preferably 200 to 600° C. A lower limit and an upper limit of the temperature of the primary calcination are more preferably 250° C. or higher and 450° C. or lower, respectively. The duration of the primary calcination is preferably 0.5 to 5 hours. The primary calcination may be performed with the dry product being immobilized using, for example, a box-type calcination furnace or a tunnel-type calcination furnace, or may be performed while allowing the dry product to flow using a rotary kiln or the like.

The temperature of the secondary calcination is preferably 300 to 700° C. A lower limit and an upper limit of the temperature of the secondary calcination are more preferably 400° C. or higher and 600° C. or lower, respectively. The duration of the secondary calcination is preferably 10 minutes to 10 hours, and a lower limit thereof is more preferably 1 hour or longer. The secondary calcination may be performed with a molded product or primary calcination product being immobilized using, for example, a box-type calcination furnace or a tunnel-type calcination furnace, or may be performed while allowing the molded product to flow using a rotary kiln or the like.

### <Molding Step>

In the molding step, a molded product is obtained by molding the dry product before or after the calcination. A molding method is not particularly limited, and any ordinary powder molding machine, such as a tablet molding machine, an extrusion molding machine, or a tumbling granulator, can be used.

At the time of the molding, a conventionally known additive may be added as well. Examples of the additive include: organic compounds, such as polyvinyl alcohol and carboxymethylcellulose; inorganic compounds, such as graphite and diatomaceous earth; and inorganic fibers, such as glass fibers, ceramic fibers, and carbon fibers.

The molded product may have any shape, such as a spherical shape, a cylindrical shape, a ring shape, a star shape, or a granular shape obtained by post-molding pulverization and classification. The molded product preferably has an outer diameter of 0.01 to 2 cm after the calcination. When the outer diameter is 0.01 cm or more, an α,β-unsaturated aldehyde and/or an α,β-unsaturated carboxylic acid can be stably produced over an extended period. Meanwhile, when the outer diameter is 2 cm or less, the strength of the molded product can be maintained. A lower limit of the outer diameter is more preferably 0.05 cm or more, still more preferably 0.1 cm or more. An upper limit of the outer diameter is more preferably 1.5 cm or less, still more preferably 1 cm or less.

The molded product preferably has an outer surface area of 0.01 to 4 cm² after the calcination. When the outer surface area is 0.01 cm² or larger, an α,β-unsaturated aldehyde and/or an α,β-unsaturated carboxylic acid can be stably produced over an extended period. Meanwhile, when the outer surface area is 4 cm² or smaller, the selectivity of an α,β-unsaturated aldehyde and/or an α,β-unsaturated carboxylic acid is improved. A lower limit of the outer surface area is more preferably 0.05 cm² or larger, still more preferably 0.1 cm² or larger. An upper limit of the outer surface area is more preferably 3 cm² or smaller, still more preferably 2 cm² or smaller.

The molded product preferably has a volume of 0.0002 to 5 cm³ after the calcination. When the volume is 0.0002 cm³ or more, an α,β-unsaturated aldehyde and/or an α,β-unsaturated carboxylic acid can be stably produced over an extended period. Meanwhile, when the volume is 5 cm³ or less, the selectivity of an α,β-unsaturated aldehyde and/or an α,β-unsaturated carboxylic acid is improved. A lower limit of the volume is more preferably 0.002 cm³ or more, still more preferably 0.02 cm³ or more. An upper limit of the volume is more preferably 1 cm³ or less, still more preferably 0.5 cm³ or less.

The molded product preferably has a mass of 0.002 to 0.5 g/product after the calcination. When the mass is 0.002 g/product or more, an α,β-unsaturated aldehyde and/or an α,β-unsaturated carboxylic acid can be stably produced over an extended period. Meanwhile, when the mass is 0.5 g/product or less, the selectivity of an α,β-unsaturated aldehyde and/or an α,β-unsaturated carboxylic acid is improved. A lower limit of the mass is more preferably 0.01 g/product or more, still more preferably 0.05 g/product or more. An upper limit of the mass is more preferably 0.3 g/product or less, still more preferably 0.2 g/product or less.

The molded product preferably has a packed bulk density of 0.2 to 1 g/cm³ after the calcination. When the packed bulk density is 0.2 g/cm³ or higher, an α,β-unsaturated aldehyde and/or an α,β-unsaturated carboxylic acid can be stably produced over an extended period. Meanwhile, when the packed bulk density is 1 g/cm³ or lower, the selectivity of an α,β-unsaturated aldehyde and/or an α,β-unsaturated carboxylic acid is improved. A lower limit of the packed bulk density is more preferably 0.3 g/cm³ or higher, still more preferably 0.4 g/cm³ or higher. An upper limit of the packed bulk density is more preferably 0.9 g/cm³ or lower, still more preferably 0.8 g/cm³ or lower. It is noted here that the packed bulk density of the molded product refers to a value calculated from a total mass of the molded product when filled into a 100-ml graduated cylinder by the method according to JIS K7365.

The resulting molded product may be supported on a carrier. Examples of the carrier used for supporting the molded product thereon include inert substances, such as silica, alumina, silica-alumina, magnesia, titania, and silicon carbide. Further, the molded product may be diluted with any of these inert substances before use.

[Method of Producing α,β-Unsaturated Aldehyde and/or α, β-Unsaturated Carboxylic Acid]

The method of producing an α, β-unsaturated aldehyde and/or an α,β-unsaturated carboxylic acid according to another embodiment of the present invention is a method which includes the step of oxidizing a hydrocarbon in the presence of the above-described catalyst, and in which, by this oxidation, a corresponding α,β-unsaturated aldehyde and/or α,β-unsaturated carboxylic acid is produced. Further, the method of producing an α, β-unsaturated aldehyde and/or an α,β-unsaturated carboxylic acid according to yet another embodiment is a method which includes the step of oxidizing a hydrocarbon in the presence of a catalyst produced by the above-described method, and in which, by this oxidation, a corresponding α,β-unsaturated aldehyde and/or α,β-unsaturated carboxylic acid is produced. According to these methods, an α, β-unsaturated aldehyde and/or an α,β-unsaturated carboxylic acid can be obtained with a high selectivity.

As the hydrocarbon, specifically, a raw material organic compound such as propylene, isobutylene, TBA, or MTBE can be used. The α, β-unsaturated aldehyde corresponding to propylene is acrolein, and the α,β-unsaturated carboxylic acid corresponding to propylene is acrylic acid. Further, the α, β-unsaturated aldehyde corresponding to isobutylene is methacrolein, and the α,β-unsaturated carboxylic acid corresponding to isobutylene is methacrylic acid. The α, β-unsaturated aldehyde corresponding to TBA is methacrolein, and the α, β-unsaturated carboxylic acid corresponding to TBA is methacrylic acid. The α, β-unsaturated aldehyde corresponding to MTBE is methacrolein, and the α, β-unsaturated carboxylic acid corresponding to MTBE is methacrylic acid. From the standpoint of improving the yield of a product, the α,β-unsaturated aldehyde and the α,β-unsaturated carboxylic acid are preferably methacrolein and methacrylic acid, respectively.

The method of producing an α, β-unsaturated aldehyde and/or an α,β-unsaturated carboxylic acid according to the present embodiment can be carried out by bringing the above-described catalyst into contact with a raw material gas containing a hydrocarbon and oxygen in a reactor. As the reactor, any reactor that is generally used for gas-phase oxidation can be used, and it is preferred to use a tube reactor equipped with a reaction tube to which the catalyst is loaded. Industrially, it is preferred to use a multi-tube reactor equipped with a plurality of such reaction tubes.

The concentration of the hydrocarbon in the raw material gas is not particularly limited; however, it is preferably 1 to 20% by volume, and it is more preferred that a lower limit thereof be 3% by volume or higher, and an upper limit thereof be 10% by volume or lower.

An oxygen source of the raw material gas is not particularly limited; however, it is industrially advantageous to use air. If necessary, a gas obtained by mixing air or the like with pure oxygen can be used as well. A ratio of oxygen in the raw material gas is not particularly limited; however, it is preferably 10 to 500% by volume with respect to the hydrocarbon, and it is more preferred that a lower limit thereof be 50% by volume or more, and an upper limit thereof be 300% by volume or less. It is noted here that, from the economic standpoint, the raw material gas is preferably used after being diluted with an inert gas such as nitrogen or carbon dioxide, water vapor, or the like.

A contact time between the raw material gas and the catalyst is not particularly limited; however, it is preferably 0.5 to 10 seconds, and it is more preferred that a lower limit thereof be 1 second or longer, and an upper limit thereof be 5 seconds or shorter. The pressure during the oxidation reaction is usually the atmospheric pressure to about several times of the atmospheric pressure. The temperature during the oxidation reaction is preferably 200 to 450° C., and it is more preferred that a lower limit thereof be 250° C. or higher, and an upper limit thereof be 400° C. or lower.

### [Method of Producing α,β-Unsaturated Carboxylic Acid]

The method of producing an α, β-unsaturated carboxylic acid according to yet another embodiment of the present invention is a method which includes the step of oxidizing an α,β-unsaturated aldehyde produced by the above-described method, and in which, by this oxidation, a corresponding α,β-unsaturated carboxylic acid is produced. From the standpoint of improving the yield of a product, the α,β-unsaturated aldehyde and the α,β-unsaturated carboxylic acid are preferably methacrolein and methacrylic acid, respectively.

The method of producing an α, β-unsaturated carboxylic acid according to the present embodiment can be carried out by, in a reactor, bringing a catalyst for the production of an α,β-unsaturated carboxylic acid into contact with a raw material gas containing an α,β-unsaturated aldehyde. As the catalyst, a heteropolyacid catalyst is preferably used. As the reactor, the same reactor as the one used in the above-described method of producing an α,β-unsaturated aldehyde can be used.

The concentration of the α, β-unsaturated aldehyde in the raw material gas is not particularly limited; however, it is preferably 1 to 20% by volume, and it is more preferred that a lower limit thereof be 3% by volume or higher, and an upper limit thereof be 10% by volume or lower.

An oxygen source of the raw material gas is not particularly limited; however, it is industrially advantageous to use air. If necessary, a gas obtained by mixing air or the like with pure oxygen can be used as well. A ratio of oxygen in the raw material gas is not particularly limited; however, it is preferably 40 to 400% by volume with respect to the α,β-unsaturated aldehyde, and it is more preferred that a lower limit thereof be 50% by volume or more, and an upper limit thereof be 300% by volume or less. It is noted here that, from the economic standpoint, the raw material gas is preferably used after being diluted with an inert gas such as nitrogen or carbon dioxide, water vapor, or the like.

A contact time between the raw material gas and the catalyst for the production of an α,β-unsaturated carboxylic acid is not particularly limited; however, it is preferably 1.5 to 15 seconds. The pressure during the oxidation reaction is usually the atmospheric pressure to about several times of the atmospheric pressure. The temperature during the oxidation reaction is preferably 200 to 400° C., and a lower limit thereof is more preferably 250° C. or higher.

### [Method of Producing α,β-Unsaturated Carboxylic Acid Ester]

The method of producing an α, β-unsaturated carboxylic acid ester according to yet another embodiment of the present invention is a method which includes the step of esterifying an α,β-unsaturated carboxylic acid produced by the above-described method, and in which, by this esterification, an α,β-unsaturated carboxylic acid ester is produced. An alcohol to be reacted with the α, β-unsaturated carboxylic acid is not particularly limited, and examples thereof include methanol, ethanol, propanol, isopropanol, butanol, and isobutanol. Examples of the resulting α, β-unsaturated carboxylic acid ester include methyl (meth)acrylate, ethyl (meth)acrylate, propyl (meth)acrylate, isopropyl (meth)acrylate, butyl (meth)acrylate, and isobutyl (meth)acrylate. The esterification reaction can be performed in the presence of an acidic catalyst, such as a sulfonic acid-type cation exchange resin. The temperature during the esterification reaction is preferably 50 to 200° C.

### EXAMPLES

Production Examples of the catalyst according to one embodiment of the present invention and Reaction Examples using the catalyst will now be described along with Comparative Examples. In the below-described Examples and Comparative Examples, “part(s)” means part(s) by mass.

### (Composition Ratio of Catalyst)

The atomic ratio of each element was determined by analyzing components of a catalyst dissolved in hydrochloric acid by ICP emission spectrometry.

### (Measurement of Bismuth Concentration of Catalyst)

The bismuth concentration of a catalyst was measured as follows.

First, a reflected electron image of the catalyst was obtained using S-3400N manufactured by Hitachi, Ltd. as an SEM apparatus. As for the measurement conditions, the accelerating voltage was set at 15 kV, the current value was set at 60 mA, and the trap time at each measurement spot and in each measurement area was set at 300 seconds. Further, the magnification was set at ×1,000.

The thus obtained reflected electron image in a bitmap format was opened in Photos manufactured by Microsoft Corporation, and an editing operation of adjusting the light to −80 was repeated three times. The resulting image was opened in Paint manufactured by Microsoft Corporation, saved as a monochromatic image, and thereby binarized into black and white.

Subsequently, the image thus binarized into black and white was analyzed by EDS to measure the bismuth concentration. As for the EDS measurement conditions, the accelerating voltage was set at 15 kV, and the current value was set at 60 mA. Measured elements were those elements (including bismuth) that had a greater number of moles than bismuth in the composition of the catalyst excluding oxygen element. Further, the analysis time per measurement spot was set at 300 seconds or longer such that the count number of all measured elements was 5,000 or more and the count number of bismuth was 10,000 or more. In the image binarized into black and white, 10 spots of white parts were selected and point-analyzed, and x2 was calculated from an average value of the 10 spots. Further, with regard to black parts, an arbitrary spot was selected in the vicinity of the respective white parts measured just before, and 10 of these spots were point-analyzed in the same manner, after which x1 and σ1 were calculated from an average value and a standard deviation of the 10 spots.

### (Measurement of X-Ray Diffraction Pattern of Catalyst)

For the measurement of an X-ray diffraction pattern of a catalyst, an X-ray diffractometer X'Pert Pro MRD manufactured by Malvern Panalytical Ltd. was used. The measurement was performed using CuKα rays (A=0.154 nm) at an output of 45 kV and 40 mA in a measurement range of 5° to 60°. The thus obtained X-ray diffraction pattern was corrected using a straight line connecting 2θ=20° and 2θ=50.2° as a baseline, and the values of IB/IA and IC/IA were calculated.

### (Evaluation of Reaction)

A reaction of each catalyst of Examples and Comparative Examples was evaluated taking the production of methacrolein and methacrylic acid by oxidation of isobutylene as an example. An analysis in the evaluation of reaction was performed by gas chromatography (apparatus: GC-2014 manufactured by Shimadzu Corporation, column: DB-FFAP manufactured by J&W Scientific, Inc., 30 m×0.32 mm; membrane thickness=0.25 μm). From the results of this gas chromatography, the selectivity of generated methacrolein and methacrylic acid was calculated by the following equation:

$$Total selectivity ( \% ) of methacrolein and methacrylic acid = ( ( Number of moles of generated methacrolein + Number of moles of generated methacrylic acid ) \text{} / Number of moles of reacted isobutylene ) ) \times 100$$

### Example 1

A liquid A was prepared by mixing 2,000 parts by mass of 60° C. pure water with 500 parts by mass of ammonium paramolybdate tetrahydrate, 23.0 parts by mass of cesium nitrate, 49.5 parts by mass of bismuth oxide, and 24.1 parts by mass of antimony trioxide. Further, a liquid B was prepared by mixing 1,000 parts by mass of pure water with 228.8 parts by mass of iron (III) nitrate nonahydrate and 480.8 parts by mass of cobalt (II) nitrate hexahydrate. Then, the liquid A and the liquid B were mixed to prepare a liquid C.

While circulating the thus obtained liquid C using a circulation pump, a 90-minute dispersion treatment of the liquid C was performed using a stirring-type homogenizer. Subsequently, the resulting dispersion-treated slurry was heated to 95° C. and maintained for 1 hour with stirring using a rotary blade stirrer to prepare a liquid D.

The thus obtained liquid D was dried using a spray dryer at an inlet temperature of 190° C. to obtain a dry product.

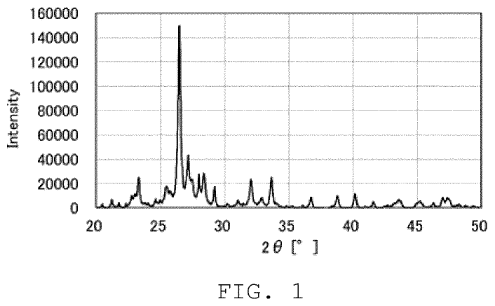

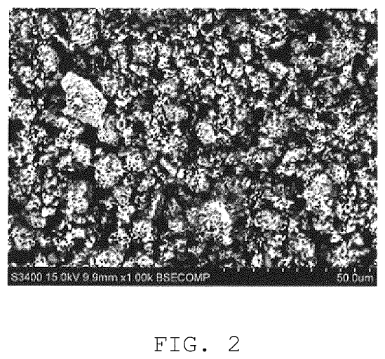

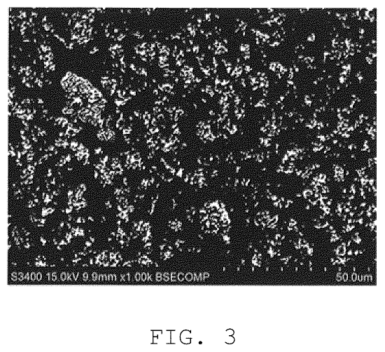

The thus obtained dry product was subjected to 1-hour primary calcination at 300° C. in an air stream. After this primary calcination, the dry product was press-molded with a pressure of 16 MPa for 20 seconds and then pulverized to obtain pulverized particles. The thus obtained pulverized particles were classified, and those particles that passed through a sieve having a mesh size of 2.36 mm but not a sieve having a mesh size of 0.71 mm were recovered to obtain a molded product in the form of granules. Thereafter, the thus obtained molded product was subjected to 6-hour secondary calcination at 500° C. in an air stream to obtain a catalyst. The thus obtained catalyst had a composition of Mo₁₂Bi_{0.90}Fe_{2.40}Co_{7.0}Sb_{0.70}Cs_{0.50}, excluding oxygen. For this catalyst, X-ray diffraction pattern and bismuth concentration were measured. The thus obtained values of IB/IA and IC/IA are shown in Table 1, and the thus measured X-ray diffraction pattern is shown in FIG. 1. Further, the thus obtained values of x1, x2, and σ1 are shown in Table 1, and a reflected electron image obtained by SEM and a binarized image thereof in black and white are shown in FIGS. 2 and 3, respectively.

The thus obtained catalyst was filled into a stainless steel reaction tube, and a raw material gas, which consisted of 5% by volume of isobutylene, 12% by volume of oxygen, 10% by volume of water vapor, and 73% by volume of nitrogen, was passed through the catalyst in the reaction tube for a contact time of 2.7 seconds to evaluate a reaction at a temperature of 330° C. The result thereof is shown in Table 1.

### Example 2

A liquid A was prepared in the same manner as in Example 1. Further, a liquid B was prepared in the same manner as in Example 1, except that the amount of cobalt (II) nitrate hexahydrate was changed to 549.5 parts by mass. Then, the liquid A and the liquid B were mixed to prepare a liquid C.

While circulating the thus obtained liquid C using a circulation pump, a 60-minute dispersion treatment of the liquid C was performed using a stirring-type homogenizer. Subsequently, the resulting dispersion-treated slurry was maintained in the same manner as in Example 1 to prepare a liquid D.

The thus obtained liquid D was dried using a spray dryer to obtain a dry product.

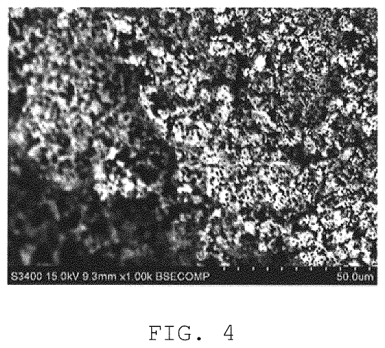

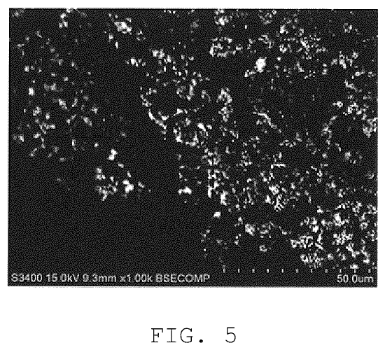

The thus obtained dry product was calcined and molded in the same manner as in Example 1 to obtain a catalyst. This catalyst had an elemental composition of Mo₁₂Bi_{0.90}Fe_{2.40}Co_{8.0}Sb_{0.70}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1, and a reflected electron image obtained by SEM and a binarized image thereof in black and white are shown in FIGS. 4 and 5, respectively.

Using the thus obtained catalyst, a reaction was evaluated in the same manner as in Example 1. The result thereof is shown in Table 1.

### Example 3

A liquid A was prepared in the same manner as in Example 1, except that the amount of antimony trioxide was changed to 34.4 parts by mass. Further, a liquid B was prepared in the same manner as in Example 1, except that the amount of iron (III) nitrate nonahydrate was changed to 171.6 parts by mass, and the amount of cobalt (II) nitrate hexahydrate was changed to 549.5 parts by mass. Then, the liquid A and the liquid B were mixed to prepare a liquid C.

While circulating the thus obtained liquid C using a circulation pump, a 60-minute dispersion treatment of the liquid C was performed using a stirring-type homogenizer. Subsequently, the resulting dispersion-treated slurry was maintained in the same manner as in Example 1 to prepare a liquid D.

The thus obtained liquid D was dried using a spray dryer to obtain a dry product.

The thus obtained dry product was calcined and molded in the same manner as in Example 1 to obtain a catalyst. This catalyst had an elemental composition of Mo₁₂Bi_{0.90}Fe_{1.80}Co_{8.0}Sb_{1.00}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1.

Using the thus obtained catalyst, a reaction was evaluated in the same manner as in Example 1. The result thereof is shown in Table 1.

### Example 4

A liquid A was prepared in the same manner as in Example 1, except that the amount of antimony trioxide was changed to 34.4 parts by mass. Further, a liquid B was prepared in the same manner as in Example 1, except that the amount of iron (III) nitrate nonahydrate was changed to 286.0 parts by mass. Then, the liquid A and the liquid B were mixed to prepare a liquid C.

While circulating the thus obtained liquid C using a circulation pump, a 120-minute dispersion treatment of the liquid C was performed using a stirring-type homogenizer. Subsequently, the resulting dispersion-treated slurry was heated to 95° C. and maintained for 2 hours with stirring using a rotary blade stirrer to prepare a liquid D.

The thus obtained liquid D was dried using a spray dryer to obtain a dry product.

The thus obtained dry product was calcined and molded in the same manner as in Example 1 to obtain a catalyst. This catalyst had an elemental composition of Mo₁₂Bi_{0.90}Fe_{3.00}Co_{7.0}Sb_{0.70}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1.

Using the thus obtained catalyst, a reaction was evaluated in the same manner as in Example 1. The result thereof is shown in Table 1.

### Example 5

A liquid A was prepared in the same manner as in Example 1, except that the amount of antimony trioxide was changed to 44.7 parts by mass. Further, a liquid B was prepared in the same manner as in Example 1, except that the amount of iron (III) nitrate nonahydrate was changed to 286.0 parts by mass. Then, the liquid A and the liquid B were mixed to prepare a liquid C.

While circulating the thus obtained liquid C using a circulation pump, a 240-minute dispersion treatment of the liquid C was performed using a stirring-type homogenizer. Subsequently, the resulting dispersion-treated slurry was heated to 95° C. and maintained for 2 hours with stirring using a rotary blade stirrer to prepare a liquid D.

The thus obtained liquid D was dried using a spray dryer to obtain a dry product.

The thus obtained dry product was calcined and molded in the same manner as in Example 1 to obtain a catalyst. This catalyst had an elemental composition of Mo₁₂Bi_{0.90}Fe_{3.00}Co_{7.0}Sb_{1.30}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1.

Using the thus obtained catalyst, a reaction was evaluated in the same manner as in Example 1. The result thereof is shown in Table 1.

### Comparative Example 1

A liquid A was prepared in the same manner as in Example 1, except that the amount of bismuth oxide was changed to 33.0 parts by mass, and the amount of antimony trioxide was changed to 34.4 parts by mass. Further, a liquid B was prepared in the same manner as in Example 1, except that the amount of cobalt (II) nitrate hexahydrate was changed to 618.2 parts by mass. Then, the liquid A and the liquid B were mixed to prepare a liquid C.

The thus obtained liquid C was heated to 95° C. and maintained for 1 hour with stirring using a rotary blade stirrer. In other words, a slurry (liquid D′) was prepared without a dispersion treatment of the liquid C.

The thus obtained liquid D′ was dried using a spray dryer to obtain a dry product.

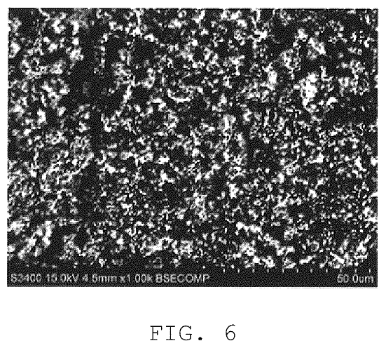

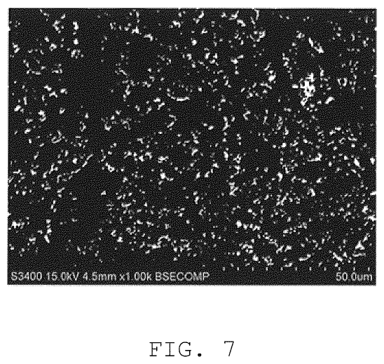

The thus obtained dry product was calcined and molded in the same manner as in Example 1 to obtain a catalyst. This catalyst had an elemental composition of Mo₁₂Bi_{0.60}Fe_{2.40}Co_{9.0}Sb_{1.00}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1, and a reflected electron image obtained by SEM and a binarized image thereof in black and white are shown in FIGS. 6 and 7, respectively.

Using the thus obtained catalyst, a reaction was evaluated in the same manner as in Example 1. The result thereof is shown in Table 1.

### Comparative Example 2

A liquid C was prepared in the same manner as in Example 1.

The thus obtained liquid C was subjected to a 90-minute dispersion treatment using a stirring-type homogenizer. In this process, the liquid C was not circulated. Subsequently, the resulting dispersion-treated slurry was maintained in the same manner as in Example 1 to prepare a liquid D.

The thus obtained liquid D was dried using a spray dryer to obtain a dry product.

The thus obtained dry product was calcined and molded in the same manner as in Example 1 to obtain a catalyst. This catalyst had an elemental composition of Mo₁₂Bi_{0.90}Fe_{2.40}Co_{7.0}Sb_{0.70}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1.

Using the thus obtained catalyst, a reaction was evaluated in the same manner as in Example 1. The result thereof is shown in Table 1.

### Comparative Example 3

A liquid A′ not containing bismuth was prepared by mixing 2,000 parts by mass of 60° C. pure water with 500 parts by mass of ammonium paramolybdate tetrahydrate. Further, a liquid B was prepared by mixing 1,000 parts by mass of pure water with 23.0 parts by mass of cesium nitrate, 49.5 parts by mass of bismuth oxide, 24.1 parts by mass of antimony trioxide, 228.8 parts by mass of iron (III) nitrate nonahydrate, and 480.8 parts by mass of cobalt (II) nitrate hexahydrate. Then, the liquid A′ and the liquid B were mixed to prepare a liquid C.

The thus obtained liquid C was dispersion-treated and maintained in the same manner as in Example 1 to prepare a liquid D.

The thus obtained liquid D was dried using a spray dryer to obtain a dry product.

The resulting dry product was calcined and molded in the same manner as in Example 1 to obtain a catalyst. This catalyst had an elemental composition of Mo₁₂Bi_{0.90}Fe_{2.40}Co_{7.0}Sb_{0.70}Cs_{0.50}, excluding oxygen. For this catalyst, X-ray diffraction pattern and bismuth concentration were measured. The thus obtained values of IB/IA and IC/IA are shown in Table 1. Further, the thus obtained values of x1, x2, and σ1 are shown in Table 1.

Using the thus obtained catalyst, a reaction was evaluated in the same manner as in Example 1. The result thereof is shown in Table 1.

TABLE 1

| | | | | | | | | Total selectivity of |
|---|---|---|---|---|---|---|---|---|
| | x1 | x2 | | | | Peak intensity | | methacrolein and |
| | [% by | [% by | | | | ratio | | methacrylic acid |
| | mass] | mass] | σ1 | x2 − x1 | (x2 − x1)/σ1 | IB/IA | IC/IA | [%] |
| Example 1 | 12.5 | 13.1 | 3.40 | 0.60 | 0.18 | 0.186 | 0.115 | 89.2 |
| Example 2 | 10.5 | 11.4 | 5.50 | 0.90 | 0.16 | 0.261 | 0.171 | 89.9 |
| Example 3 | 11.2 | 12.5 | 2.10 | 1.30 | 0.62 | 0.134 | 0.101 | 89.1 |
| Example 4 | 13.5 | 14.2 | 4.80 | 0.70 | 0.15 | 0.305 | 0.185 | 88.9 |
| Example 5 | 13.5 | 14.4 | 4.90 | 0.90 | 0.18 | 0.352 | 0.192 | 88.5 |
| Comparative | 9.5 | 15.4 | 1.50 | 5.90 | 3.93 | 0.061 | 0.029 | 87.7 |
| Example 1 | | | | | | | | |
| Comparative | 9.3 | 14.8 | 2.10 | 5.50 | 2.62 | 0.075 | 0.041 | 86.9 |
| Example 2 | | | | | | | | |
| Comparative | 9.1 | 14.1 | 2.50 | 5.00 | 2.00 | 0.072 | 0.036 | 87.1 |
| Example 3 | | | | | | | | |

As shown in Table 1, a favorable total selectivity of methacrolein and methacrylic acid was obtained in Examples 1 to 5, where a catalyst satisfying a specific bismuth concentration in a reflected electron image obtained by SEM, or a catalyst having a prescribed intensity ratio in an X-ray diffraction pattern was used.

Methacrylic acid can be obtained by oxidizing methacrolein obtained in the present Examples, and a methacrylic acid ester can be obtained by esterifying methacrylic acid.

### INDUSTRIAL APPLICABILITY

According to the present invention, a catalyst with which an α,β-unsaturated aldehyde and/or an α,β-unsaturated carboxylic acid can be produced with a high selectivity can be provided.

While the invention has been described in detail and with reference to specific embodiments thereof, it will be apparent to one skilled in the art that various changes and modifications can be made therein without departing from the spirit and scope thereof.

What is claimed is:

1. A catalyst used for producing, by an oxidation reaction of a hydrocarbon, a corresponding α,β-unsaturated aldehyde and/or α,β-unsaturated carboxylic acid,
wherein the catalyst comprises molybdenum, bismuth, and cobalt, and satisfies the following Formula (I-1):
$$( x 2 - x 1 ) / σ 1 \leq 1.5 \qquad ( I - 1 )$$
wherein, x1, x2, and σ1 are values obtained by binarizing a reflected electron image of the catalyst, which is obtained using a scanning electron microscope (SEM) at an accelerating voltage of 15 kV, into black and white and subsequently performing an energy dispersive X-ray spectroscopy (EDS) analysis; x1 represents a bismuth concentration [% by mass] in black parts; x2 represents a bismuth concentration [% by mass] in white parts; and σ1 represents a standard deviation of the bismuth concentration in the black parts.

2. The catalyst according to claim 1, wherein the x1, x2, and ad satisfy the following Formula (I-2):
$$( x 2 - x 1 ) / σ 1 \leq 1 \qquad ( \text{I-2} )$$

3. The catalyst according to claim 1, wherein the x1 and x2 satisfy the following Formula (I-3):
$$x 2 - x 1 \leq 3.5 \qquad ( I - 3 )$$

4. The catalyst according to claim 1, wherein the σ1 is 1.5 or more.

5. The catalyst according to claim 1, wherein, in an X-ray diffraction pattern, when the intensity of a maximum peak (peak A) in a range of 2θ (X-ray diffraction angle)=26.5°±0.3° is defined as IA and the intensity of a maximum peak (peak B) in a range of 2θ=28.0°±0.3° is defined as IB, a value of IB/IA is 0.08 or more.

6. The catalyst according to claim 5, wherein the value of IB/IA is 0.08 to 0.5.

7. A catalyst used for producing, by an oxidation reaction of a hydrocarbon, a corresponding α,β-unsaturated aldehyde and/or α,β-unsaturated carboxylic acid,
wherein
the catalyst comprises molybdenum, bismuth, and cobalt, and
in an X-ray diffraction pattern, when the intensity of a maximum peak (peak A) in a range of 2θ (X-ray diffraction angle)=26.5°±0.3° is defined as IA and the intensity of a maximum peak (peak B) in a range of 2θ=28.0°±0.3° is defined as IB, a value of IB/IA is 0.08 to 0.5.

8. The catalyst according to claim 6, wherein the value of IB/IA is 0.13 to 0.3.

9. The catalyst according to claim 1, wherein, in an X-ray diffraction pattern, when the intensity of a maximum peak (peak A) in a range of 2θ (X-ray diffraction angle)=26.5°±0.3° is defined as IA and the intensity of a maximum peak (peak C) in a range of 2θ (X-ray diffraction angle)=29.1°±0.3° is defined as IC, a value of IC/IA is 0.05 or more.

10. The catalyst according to claim 1, having a composition represented by the following Formula (II):
MoₐBi_{b}Fe_{c}Co_{d}XₑY_{f}Si_{g}Oₕ (II)
wherein, Mo, Bi, Fe, Co, Si, and O represent molybdenum, bismuth, iron, cobalt, silicon, and oxygen, respectively; X represents at least one element selected from the group consisting of nickel, calcium, magnesium, niobium, tungsten, antimony, phosphorus, and titanium; Y represents at least one element selected from the group consisting of cesium, lithium, sodium, potassium, rubidium, and thallium; a, b, c, d, e, f, g, and h represent atomic ratios of the respective elements; a=12, b=0.01 to 3, c=0 to 5, d=1 to 12, e=0 to 8, f=0.001 to 2, and g=0 to 20; and h represents an atomic ratio of oxygen that is required for satisfying the valences of the respective elements.

11. A catalyst production method, which is a method of producing the catalyst according to claim 1, and comprises the following steps (i) to (iii):
(i) the step of mixing a solution or slurry (liquid A) comprising molybdenum and bismuth with a solution or slurry (liquid B) comprising cobalt to prepare a slurry (liquid C);
(ii) the step of performing a dispersion treatment of the liquid C to prepare a slurry (liquid D); and
(iii) the step of drying the liquid D to obtain a dry product.

12. The catalyst production method according to claim 11, wherein, in the step (ii), the liquid C is circulated for 5 minutes to 10 hours during the dispersion treatment of the liquid C.

13. The catalyst production method according to claim 12, wherein, in the step (ii), the liquid C is circulated using a circulation pump.

14. The catalyst production method according to claim 11, wherein, in the step (ii), the liquid D is prepared by maintaining the resulting slurry at 60° C. or higher for at least 20 minutes after the dispersion treatment of liquid C.

15. A method of producing an α, β-unsaturated aldehyde and/or an α,β-unsaturated carboxylic acid, the method comprising the step of oxidizing a hydrocarbon in the presence of the catalyst according to claim 1.

16. A method of producing an α, β-unsaturated aldehyde and/or an α,β-unsaturated carboxylic acid, the method comprising the step of oxidizing a hydrocarbon in the presence of a catalyst produced by the method according to claim 11.

17. A method of producing an α, β-unsaturated carboxylic acid, the method comprising the step of oxidizing an α,β-unsaturated aldehyde produced by the method according to claim 15.

18. A method of producing an α, β-unsaturated carboxylic acid ester, the method comprising the step of esterifying an α,β-unsaturated carboxylic acid produced by the method according to claim 17.
